(19) United States
(12) Patent Application Publication
(10) Pub. No.: US 2014/0213466 A1
(43) Pub. Date: Jul. 31, 2014

(54) HIGH-THROUGHPUT ASSESSMENT METHOD FOR CONTACT HYPERSENSITIVITY

(71) Applicants: Rene S. Schloss, East Brunswick, NJ (US); Martin L. Yarmush, Newton, MA (US); Tim Maguire, Piscataway, NJ (US); Dave Xu Dong, South Plainfield, NJ (US); Serom Lee, Somerset, NJ (US)

(72) Inventors: Rene S. Schloss, East Brunswick, NJ (US); Martin L. Yarmush, Newton, MA (US); Tim Maguire, Piscataway, NJ (US); Dave Xu Dong, South Plainfield, NJ (US); Serom Lee, Somerset, NJ (US)

(73) Assignee: Rutgers, The State University of New Jersey, New Brunswick, NJ (US)

(21) Appl. No.: 13/988,274

(22) PCT Filed: Nov. 18, 2011
(86) PCT No.: PCT/US11/61427

§ 371 Date: Jan. 6, 2014

Related U.S. Application Data
(60) Provisional application No. 61/415,652, filed on Nov. 19, 2010.

(51) Int. Cl. G01N 33/50; G01N 33/68

(52) U.S. Cl. CPC G01N 33/5023; G01N 33/6863; G01N 33/5044; G01N 33/5029
USPC 506/9; 435/34; 435/7.21; 506/10; 702/19

(57) ABSTRACT

The present invention provides methods for high-throughput assessment of in vivo skin sensitizing activity of chemical compounds through detection of secretion levels of cytokine markers implicated in skin sensitization in combination with a multivariate analysis, using support vector machine (SVM) for feature selection. The invention includes a computational algorithm that will provide unbiased analysis on the skin cell secretome data and predict the level of skin sensitization. The invention allows accurate assessment of the level sensitizing potency of any chemicals in a high-throughput manner, which can eliminate the needs for animal experiments, potentially saving money and time.

### CROSS-REFERENCE TO RELATED APPLICATIONS

This application claims priority under 35 U.S.C. §119(e) to U.S. Provisional Patent Application Ser. No. 61/415,652, filed on Nov. 19, 2010, which is hereby incorporated by reference in its entirety.

### FIELD OF THE INVENTION

The present invention relates to methods for high-throughput assessment of sensitizing activity of chemical compounds and techniques to measure post-sensitization skin or artificial skin/dentric cell co-cultured metrics that can be used to predict and classify sensitizer potency.

### BACKGROUND OF THE INVENTION

A substance is classified as a skin sensitizer when it can induce a sensitization response following skin contact in a substantial number of persons or when there are positive results from an appropriate animal test. Therefore, the prediction of the sensitizing capacity of a chemical is of importance to the chemical, pharmaceutical, cosmetic and personal care industries. In fact, these industries are required to identify hazards and evaluate potential risks of new drugs and personal care products to consumers. Although recent legislation has limited or banned the use of animals for safety testing of products classified as cosmetics, most of currently used methods in the art for predicting sensitizing potency still involve animal testing.

Various existing in vitro methods for predicting sensitizing activity of compounds, such as the murine local lymph node assay (LLNA) and the guinea pig maximization test (GPMT), and their drawbacks have been well documented. See, e.g., McKim, J. M., US 2009/0305276; Yuan, H., et al., Int. J. Mol. Sci., 2009, 10(7), 3237-3254, and references cited therein, which are hereby incorporated by reference.

Contact hypersensitivity results from direct exposure to a large number of natural and synthetic chemicals and is initiated by dendritic cell (DC) maturation which ultimately activates allergen specific T cell responses. DC maturation criteria include an array of cell surface molecular expression changes, an array of unique cytokine secretion profile changes and activation of specific T cell subpopulations. For example, different cytokines are important in instructing T-cell differentiation to Th1-cells (IL-12, IL-27, IFNg), Th2-cells (IL-4, IL-5, IL-6, GM-CSF, PGE2), or T-regulatory cells (IL-10, TGF-β). In addition, cytokines are secreted both by DC and cells such as keratinocytes and fibroblasts which are contained in skin. While Th2 cells are associated with IgE and eosinophil mediated allergy effects, TH1 cells are associated with CD8 T cell mediated effects which accompany some hypersensitivity responses and regulatory T cells attenuate both types of responses.

It is currently unclear which of the large array of DC and/or T cell maturational changes are critical to inducing hypersensitivity and, thus, the current methods of in vitro assessment may not accurately predict in vivo responses. Therefore, there remains a need to develop new methods for assessment of chemical compounds for their sensitizing activities, in particular a method that could be conducted in a high-throughput and economic manner

### SUMMARY OF THE INVENTION

This invention provides a novel approach to fulfill the foregoing need and provides a method for high-throughput assessment and classification of chemical compounds for their sensitizing activity, by combining detection of cytokines in an in vitro cell model implicated in skin sensitization with a multivariate analysis using a computational algorithm. The computational algorithm provides unbiased analysis on the skin cell secretome data and predicts the level of skin sensitization. The invention will allow a person of skill in the art to accurately assess the level sensitizing potency of any chemicals in a high-throughput manner, which will eliminate the need for animal experiments, potentially saving money and time.

In particular, the present invention provides a computational approach and a technique that measures post-sensitization skin and dendritic cell co-culture metrics (e.g. cytokine profiles, chemotaxis, and cell surface expression) and predicts and classifies sensitizer potency. This was accomplished by using support vector machine (SVM) as a feature selection method, which aims to construct a decision boundary that would maximize the marginal distance between the two classes of chemicals (sensitizing vs. non-sensitizing) in each one of the aforementioned co-culture metrics. Chemicals that elicit a drastic change in response would have a highly different profile in one or more of the metrics. One would then be able to rank the metrics based on a score, which is the product of their class separation (i.e., the distance by which the data points lie near the decision boundary) and accuracy (i.e., how accurate is the decision boundary in separating the classes).

Thus, in one aspect the present invention provides a method for assessing potency of skin sensitizers, comprising measuring one or more co-culture metrics of post-sensitization skin or artificial skin/dentric cells; analyzing the co-culture metrics using a computational algorithm; and comparing the analysis results with those of a set of known sensitizers.

In another aspect the present invention provides a method of assessing in vivo skin sensitizing activity of a compound, including the steps of: (a) culturing cells of an in vitro cell model in a medium; (b) adding a test compound at a concentration to the culture medium comprising the cells; (c) measuring secretion level of one or more cytokine markers of the cells; (d) analyzing correlation of the concentration applied in step (b) with the secretion level measured in step (c); and (e) determining in vivo sensitization value based on the analysis of step (d).

In another aspect the present invention provides a method for determining the sensitizing activity of a compound, comprising: (a) incubating a test compound with cells to allow for binding; (b) measuring the amount of secreted cytokines; and (c) comparing the secretion profile of the cytokines with a training set of known sensitizers.

In another aspect the present invention provides high-throughput assessment methods by using multi-well transwell chambers for detecting the secretion of cytokines induced by compounds tested.

In another aspect the present invention provides an immune modeling system for predicting potency of skin sensitizers, the system containing: a) a viable epidermis to provide barrier function and skin metabolism; and b) a dendritic cell compartment, wherein dendritic cells are activated.

In addition, the present invention is be used to aid the design of experiments to narrow down on the potential metric of interest for further testing. This approach will be critical in developing better in vitro approaches to circumvent animal testing and will be important in both the cosmetic and pharmaceutical industries in assessing the efficacy/side effect potential of topical products. Additional embodiments and advantages and other aspects of the present invention will be readily apparent to one of skill in the art, based on the teaching provided herein.

### BRIEF DESCRIPTION OF THE DRAWINGS

FIG. 1 is a schematic representation of eosinophil: (A) maturation, (B) migration, (C) activation, and (D) pleiotrophic actions during allergic immune responses. Eosinophils represent one allergy response endpoint effector that is activated by T cells.

FIG. 2 illustrates control of allergic reactions by cytokines. Both Th1 and Th2 type cells may participate in allergic hypersensitivity responses, although the majority appear to be Th2 mediated, and T cell subpopulations, via cytokines, can be regulated and also regulate other cell types.

FIG. 3 illustrates secretion of cytokines by dendritic cells (DC). Cytokines and downstream transcription factors are important in instructing T-cell differentiation to Th1-cells (IL-12, IL-27, IFNγ), Th2-cells (IL-4, IL-5,IL-6, GM-CSF, PGE₂), or T-regulatory cells (IL-10, TGF-β). Cytokines are secreted both by DC and cells such as keratinocytes and fibroblasts. Th2 cells are associated with IgE and eosinophil mediated allergy effects, and Th1 cells are associated with CD8 T cell mediated effects which accompany some hypersensitivity responses. Regulatory T cells attenuate both types of responses.

FIGS. 4A and 4B illustrate cytokine and growth factor secretion profiles of Mutz3 phenotypes, which include the profiles of a portion of the 27 cytokines/growth factors studied in the present invention.

FIG. 5 illustrates IL-8 secretion by Mutz3 phenotypes.

FIG. 6 illustrates feature importance generated by data bagging.

FIG. 7 depicts a pruned decision tree generated to classify inputs, in which input of 25 features resulted in output of 4 classes (IE 150 ug, SA 100 ug, PPD 40 ug, Untreated). Hu IL-1b and Hu IL-1ra were determined to be decisive classifying features.

FIG. 8 illustrates comparison to the pruned tree on FIG. 7. Features 1 and 2 (Hu IL-1b and Hu IL-1ra) were decisive enough that the remaining features were not needed for classification.

FIG. 9 illustrates error quantification over number of trees. Increasing the number of trees used in ensemble averaging decreases random error—more accurate classification.

FIG. 10 illustrates a decision tree on published QSAR and LLNA data. The decision tree demonstrates that metrics such as molecular weigh (MW), skin penetration coefficient (log K), and octanol-water partition coefficient (log Ko/w) do not provide a reliable means to classify the chemicals, as indicated by the numerous branches that identify the same class.

FIG. 11 illustrates the results of principal component analysis (PCA) (a limited, traditional approach), which shows that the chemicals do not induce distinct cytokine profiles, especially between isoeugenol (IE, a strong sensitizer) and sialic acid (SA, a non-sensitizing irritant).

FIG. 12 illustrates that PCA reduces the dimension by projecting the data unto orthogonal axes that have the highest variance. This projection, however, does not take into account the difference between intra- and inter-class variance. Two of the 27 dimensions (IL6 and G-CSF) contribute to the most variance (the “high weights”); yet the inter-class difference between SA and IE is unclear.

FIG. 13 illustrates feature selection using quadratic discriminant analysis (QDA), which shows that IL6 and IL12 gave the lowest error.

FIGS. 14A & 14B illustrate hierarchical clustering of cytokine profiles from the full skin set and the reduced set of RSLC only. Hierarchical clustering groups features together based on how close they are from each other, using Eucledian distance. The clusters are ranked, with the closest features clustered in the lowest brackets, and merge as the hierarchy goes up.

FIG. 15 illustrates feature selection using support vector machine (SVM).

FIG. 16 illustrates SVM feature selection in full ranking of all 27 cytokines in different skin types. Higher margin distance implies better predicative power.

FIG. 17 illustrates use of leave-one-out cross validation to test the accuracy of the boundaries generated in SVM. The accuracy score can then be used to combine the cytokine scores to generate a weighted classifier which takes both the margin distance and accuracy into account.

### DETAILED DESCRIPTION OF THE INVENTION

The present invention provides a computational approach and technique that measures post-sensitization skin and dendritic cell co-culture metrics (e.g. cytokine profiles, chemotaxis, cell surface expression), which predicts and classifies sensitizer potency. This was accomplished by using support vector machine (SVM) as a feature selection method, which aims to construct a decision boundary that would maximize the marginal distance between the two classes of chemicals (sensitizing vs. non-sensitizing) in each one of the aforementioned co-culture metrics.

Machine learning algorithms have been used in various forms to analyze the LLNA data for skin sensitization (see Ren, Y., et al., Anal. Chim. Acta, 2006, 572(2), 272-282; Yuan, H., et al., Int. J. Mol. Sci., 2009, 10(7), 3237-3254). None of these studies have focused on the secreted products of the immuno-regulatory cells, which is an important aspect in order to accurately understand the in vivo response. When compared to other classification techniques, such as discriminant analysis, the support vector machine (SVM) has been proven advantageous in handling classification tasks in cases of high dimensionality of data points.

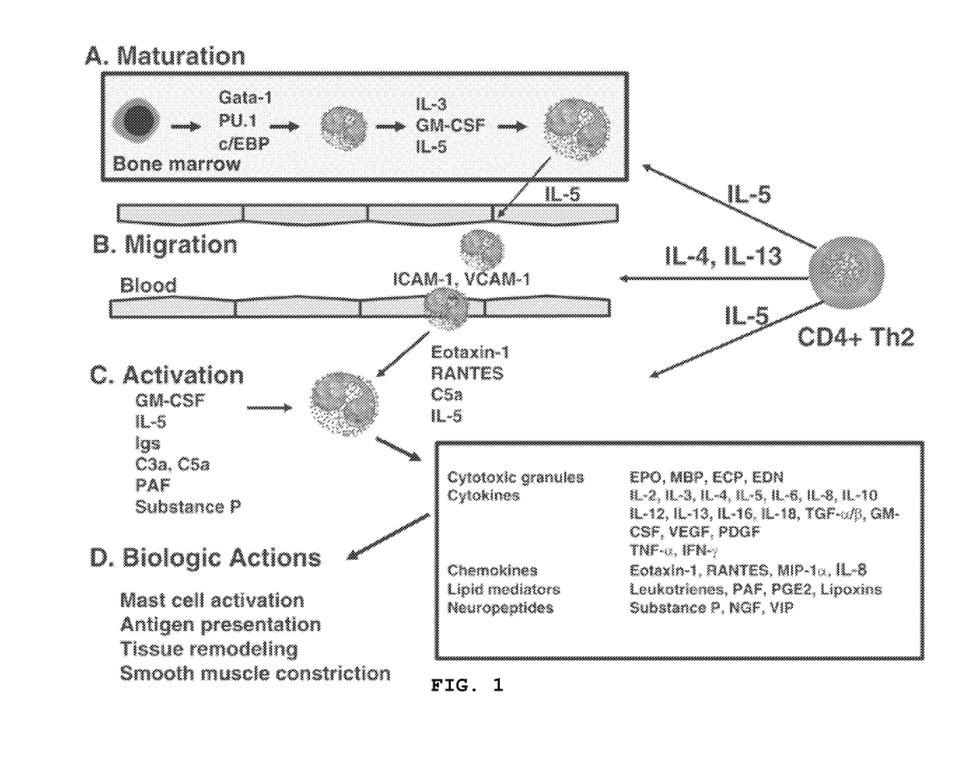

The present invention uses cytokines or growth factors as markers to determine contact hypersensitivity using an in vitro cell model, in particular Mutz3 phenotypes and EpiSkin™. A large number of cytokines control allergic responses, for example, cytotoxic granules (e.g., EPO, MBP, ECP, EON), cytokines (e.g., IL-2, IL3, IL-4, IL-5, IL-6, IL-8, IL-10, IL-12. IL-13, IL-16, IL-16, TGF-α/β, GM-CSF, VEGF, PDGF, TNF-α, IFN-γ), chemokines (e.g., Eotaxin-1, RANTES, MIP-1α, IL-8), lipid mediators (e.g., Leukotrienes, PAF, PGE₂, Lipoxins), and neuropeptides (e.g., Substance P, NGF, VIP). For schematic representation of eosinophil, see FIG. 1, which shows (A) maturation, (B) migration, (C) activation, and (D) pleiotrophic actions during allergic immune responses. Eosinophils represent one allergy response endpoint effector that is activated by T cells.

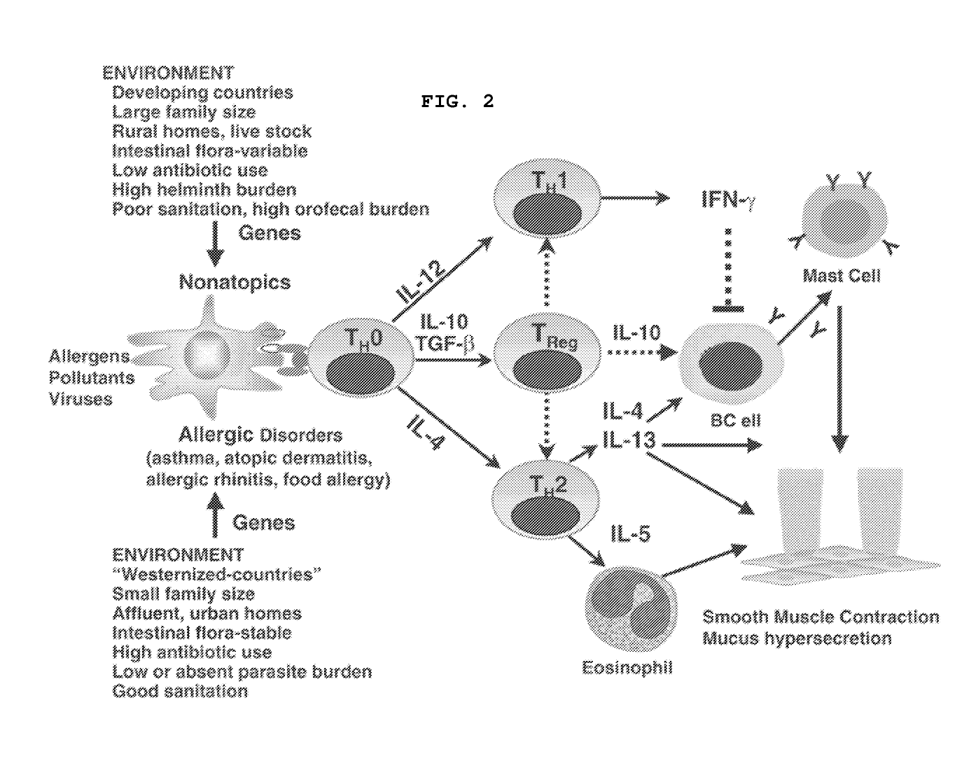

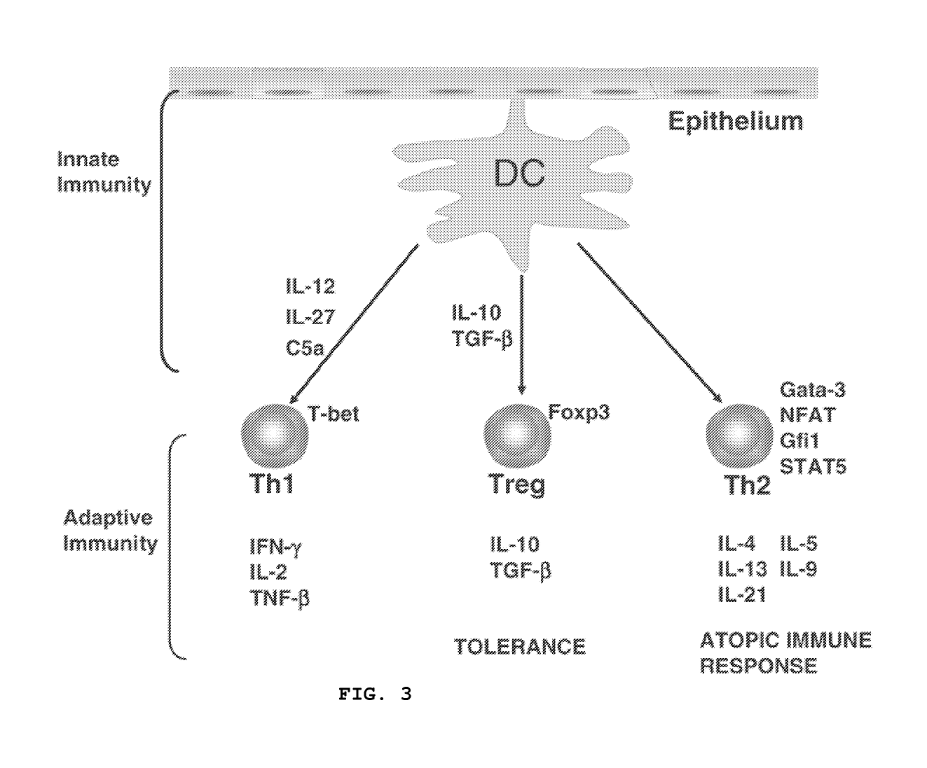

As shown in FIG. 2, both Th1 and Th2 type cells may participate in allergic hypersensitivity responses, although the majority appear to be Th2 mediated, and T cell subpopulations, via cytokines, can be regulated and also regulate other cell types. As shown in FIG. 3, cytokines and downstream transcription factors are important in instructing T-cell differentiation to Th1-cells (IL-12, IL-27, IFNγ), Th2-cells (IL-4, IL-5,IL-6, GM-CSF, PGE₂), or T-regulatory cells (IL-10, TGF-β). Cytokines are secreted both by DC and cells such as keratinocytes and fibroblasts. Th2 cells are associated with IgE and eosinophil mediated allergy effects, and Th1 cells are associated with CD8 T cell mediated effects which accompany some hypersensitivity responses. Regulatory T cells attenuate both types of responses.

While the present invention is not limited by any particular theory, the invention is based on the hypothesis that a comprehensive quantitative computational assessment of post-sensitization skin or artificial skin/DC co-culture cytokine profiles (and other metrics) may be used to predict and classify sensitizer potency. Mathematical analyses of LLNA responses and sensitizer chemical structure have been used by others to classify sensitizer potency, while the analysis used in the present invention will both independently and in concert with previous data assess the association of unique cytokine secretion patterns and other metrics over time, post-exposure to a panel of sensitizers of varying potencies and concentrations

Thus, one of the objectives of the present invention was to determine a number of metrics that are being altered temporally in response to different chemical stimuli, including but not limited to cytokine profile, cell migration, cell surface expression, and intracellular protein expression, etc. Another objective was to use multivariate analysis to determine a ranking system for the chemical compounds tested. To achieve these objectives, data acquisition systems such as the Bio-Plex suspension array system can be used, which can analyze up to 100 biomolecules in a single sample.

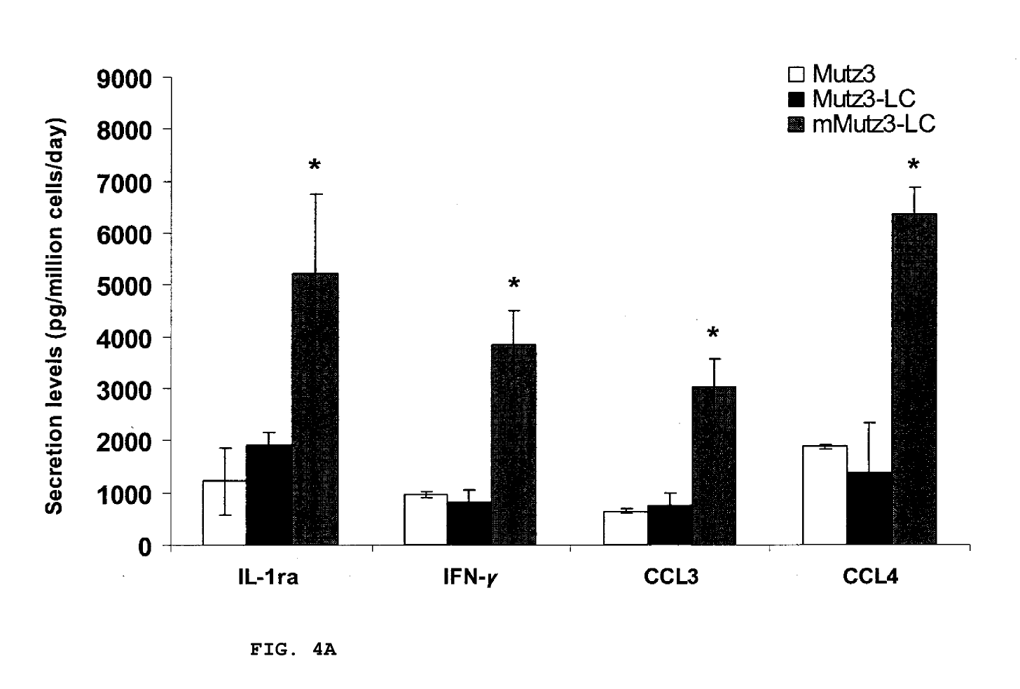

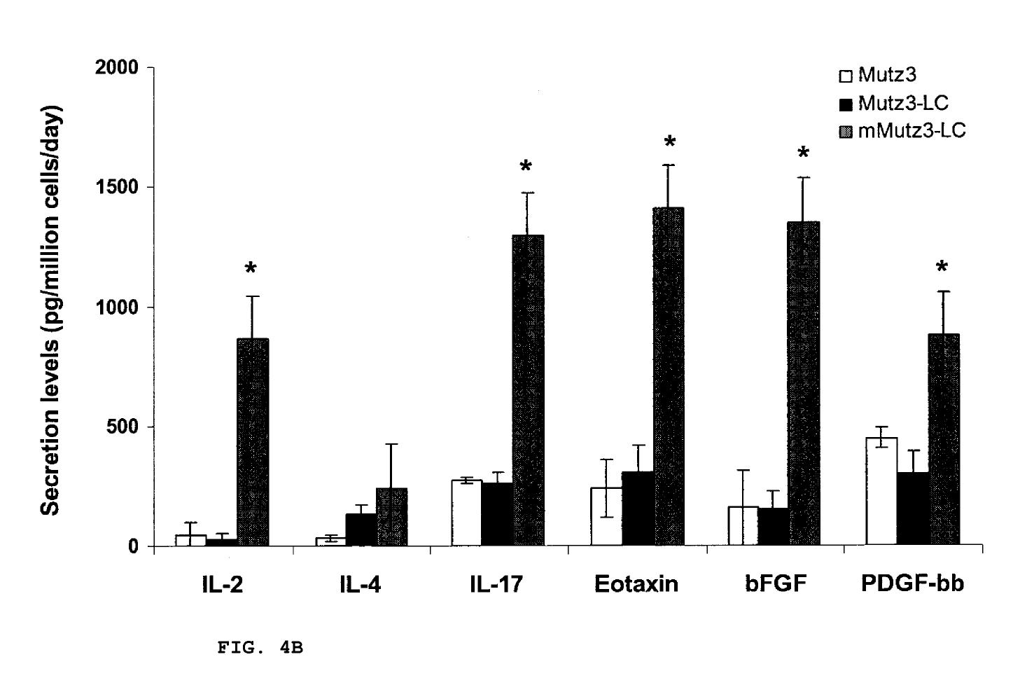

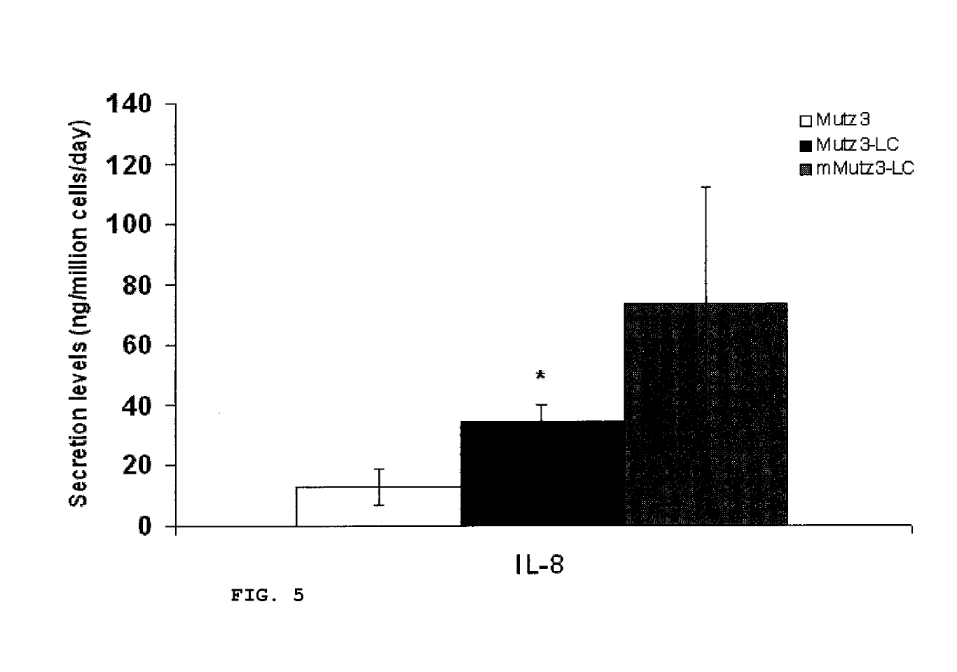

Cytokine and growth factor secretion profiles of Mutz3 phenotypes are illustrated in FIGS. 4A and 4B, which include the profiles of a portion of the 27 cytokines/growth factors studied in the present invention. In particular, as shown in FIG. 5, IL-8 Secretion by Mutz3 phenotypes is of particular importance, because IL-8 is a chemokine that induces neutrophil or T-lymphocyte migration (see, e.g., Nishiyama, N., et al., J. Toxicol. Sci., 2008, 33, 175-185). IL-8 is a commonly used DC-activation marker for in vitro discrimination of sensitizers from irritants (see, e.g., Python, F., et al., Toxicol. App./Pharmacol. 2009, 239, 273-283).

In one aspect the present invention uses multivariate analysis to create a predictive metric set utilizing the migration, cell surface, and cytokine secretome data, by using a variation of the ID3 program developed by Quinlan. ID3 builds a decision tree from a fixed set of examples, also known as the training set. The resulting tree is used to classify future samples. The leaf nodes of the decision tree contain the class name whereas a non-leaf node is a decision node. The decision node is an attribute test with each branch (to another decision tree) being a possible value of the attribute. ID3 uses a statistical property known as information gain to help it decide which attribute goes into a decision node. Gain measures how well a given attribute separates training examples into targeted classes. The one with the highest information (information being the most useful for classification) is selected. To define gain, the invention can adopt, for example, an idea from information theory called entropy, which measures the amount of information in an attribute. Using the described strategy, a training set can be derived from a known rank-order of, for example, ten pro-haptens; and the model can be tested on another set of, e.g., 20 pro-haptens, and compared to results of other tests already deployed.

Thus, in one embodiment the present invention contains a two-stage process to create a predictive sensitization metric set utilizing the migration, cell surface, and cytokine secretome data, which include (1) determining dominant parameters from a large data set, and (2) using non-linear regression to generate a metric utilizing the dominant parameter data in conjunction with an output classifier from a training set.

The model can be validated on a very large number of compounds. To implement the invention, the microfluidics device can be redesigned to maximize user interface efficiency (i.e. interface with a cell culture/liquid handling robot).

Exemplary devices/systems of the present invention include the immune modeling systems known in the art, which utilize, for example, 1) a viable epidermis to provide barrier function and skin metabolism, 2) a dendritic cell/Langerhans cell compartment within which these cells can be activated, 3) and a T cell compartment that will allow for T cell activation by migrating, activated dendritic cells (e.g., Langerhans cells). In some embodiments of the devices, systems, and methods disclosed herein, such three compartment devices are used. In some embodiments of the devices, systems, and methods disclosed herein two compartment devices are used instead. In some such embodiments, for example, the device comprises 1) a viable epidermis to provide barrier function and skin metabolism and 2) a dendritic cell/Langerhans cell compartment within which these cells can be activated, but does not comprise a T cell compartment.

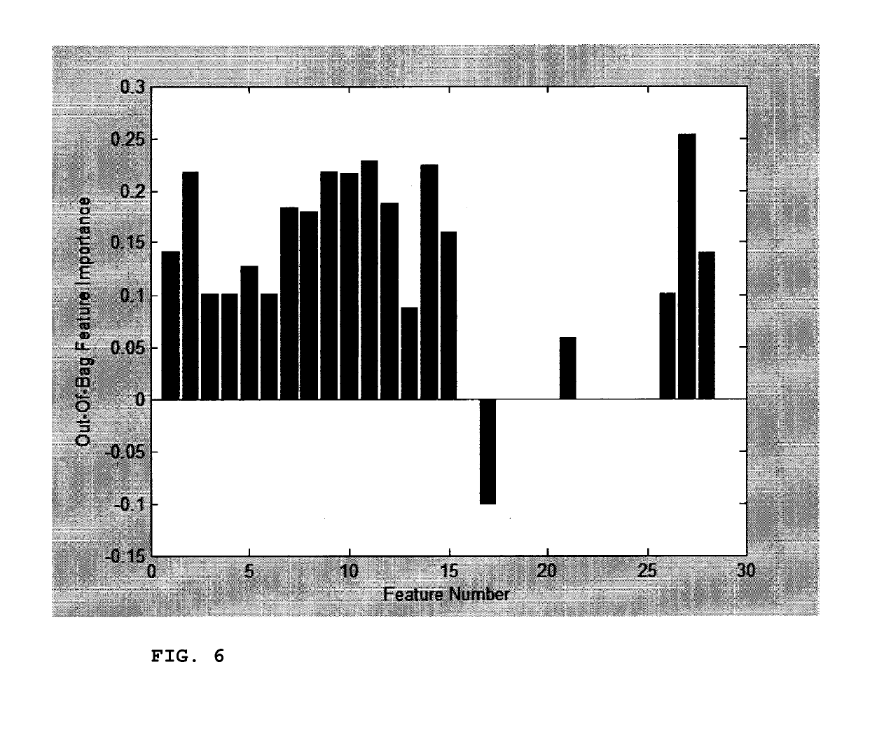

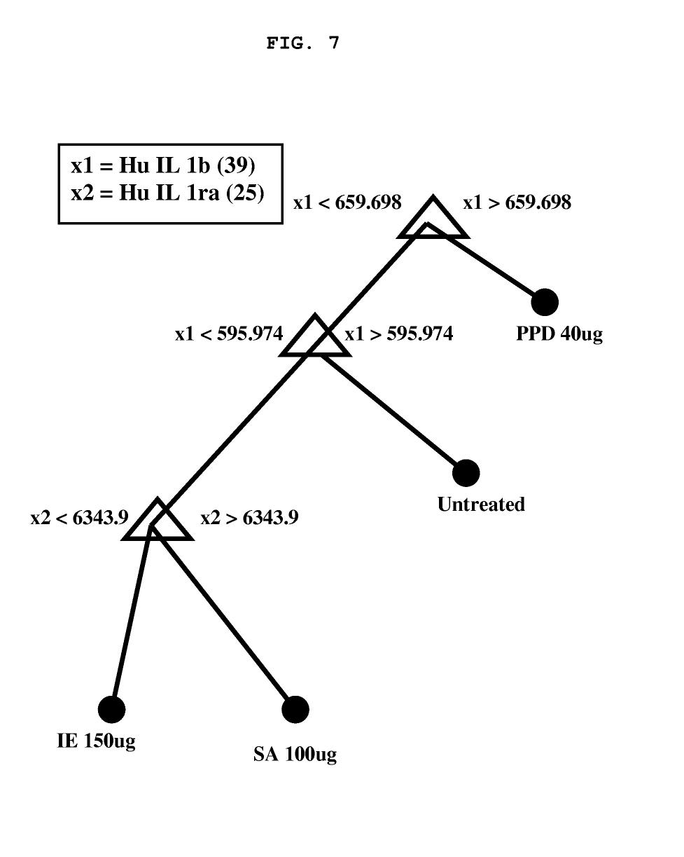

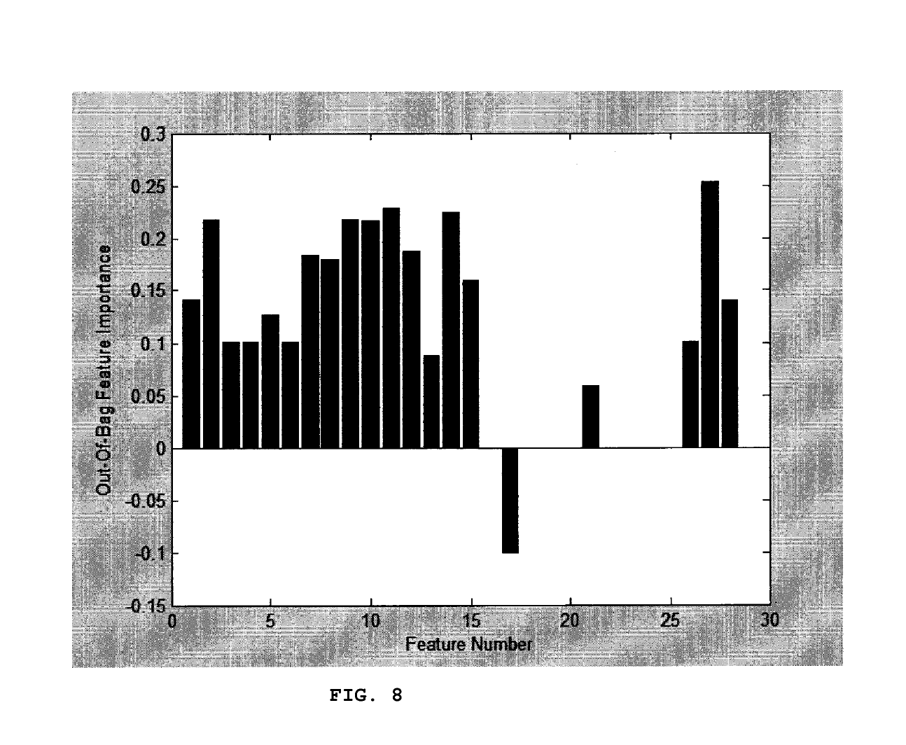

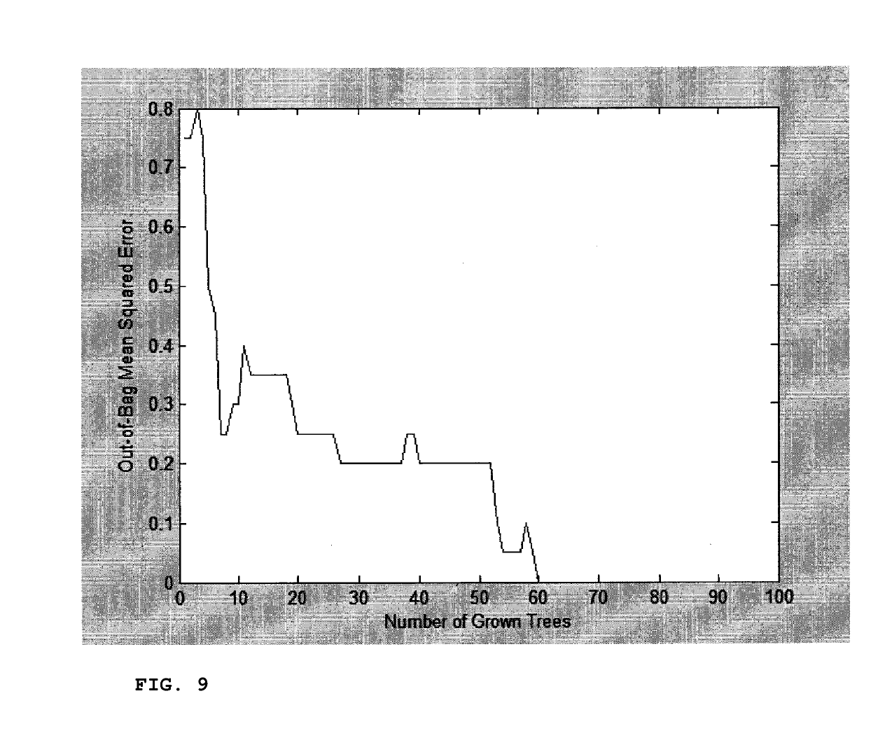

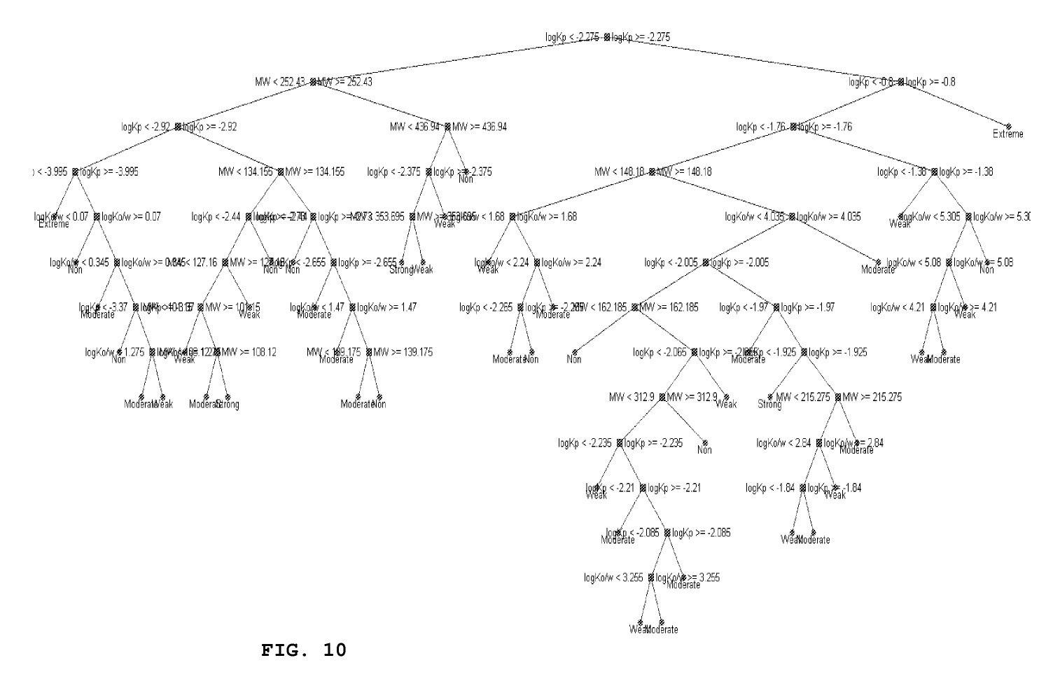

As an illustrative example, FIG. 6 shows feature importance generated by data bagging; FIG. 7 depicts a decision tree generated to classify inputs, in which input of 25 features resulted in output of 4 classes (IE 150 ug, SA 100 ug, PPD 40 ug, Untreated). Hu IL-1b and Hu IL-1ra were determined to be decisive classifying features. FIG. 8 illustrates comparison to the pruned tree on FIG. 7. Features 1 and 2 (Hu IL-1b and Hu IL-1ra) were decisive enough that the remaining features were not needed for classification. FIG. 9 illustrates error quantification over number of trees. Increasing the number of trees used in ensemble averaging decreases random error—more accurate classification.

Thus, in one aspect the present invention provides a method for assessing potency of skin sensitizers, comprising measuring one or more co-culture metrics of post-sensitization skin or artificial skin/dentric cells; analyzing the co-culture metrics using a computational algorithm; and comparing the analysis results with those of a set of known sensitizers.

In one embodiment of this aspect, the co-culture metrics are selected from cytokine profiles, chemotaxis, cell migration, cell surface expression, and intracellular protein expression.

In another embodiment of this aspect, the measuring steps includes determining the changes of the metrics in response to different chemical stimuli.

In another embodiment of this aspect, the measuring steps includes a high-throughput data acquisition through testing a plurality of stimuli compounds simultaneously.

In another embodiment of this aspect, the testing step includes use of a Bioplex suspension array system.

In another embodiment of this aspect, the measuring step includes detecting cytokine or growth factor secretion profile of a Mutz3 phenotype selected from Mutz3, Mutz3-LC, and mMutz3-LC, wherein the cytokine or growth factor controls allergic responses and is involved in contact hypersensitivity.

In another embodiment of this aspect, the cytokine or growth factor is selected from IL-1ra, IL-2, IL-4, IL-5, IL-6, IL-7, IL-8, IL-9, IL-10, IL-12, IL-13, IL-15, IL-17, eotaxin, bfGF, G-CSF, GM-CSF, IFN-γ, IP-10, MCP-1(MCAF), CCL3, CCL4, PDGF-bb, TNF-a, VEGF, and IL8.

In another embodiment of this aspect, the cytokine or growth factor is selected from IL-1ra, IFN-γ, CCL3, CCL4, IL-2, IL-4, IL-17, Eotaxin, bFGF, PDGF-bb, and IL-8.

In another embodiment of this aspect, the analyzing step includes determining a ranking system for chemical stimuli tested using a multivariate analysis.

In another embodiment of this aspect, the multivariate analysis includes the steps of:

(a) creating a predictive sensitization metric set;

(b) building a decision tree from a fixed set (also known as the “training set”) of examples;

(c) comparing attribute(s) of a test sample with the predictive metric set; and

(d) classifying the tested sample based on comparison results and the decision tree,

wherein the decision tree contains a plurality of leaf nodes and a plurality of non-leaf nodes, wherein the leaf nodes contain class names, and a non-leaf node is a decision node, the decision node being an attribute test and containing a plurality of branches, with each branch being a possible value of the attribute and connected to another decision tree.

In another embodiment of this aspect, the predictive sensitization metric set is created using an ID3 program, the program including: (i) using a statistical property known as information gain to help decide which attribute goes into a decision node, and (ii) defining information gain using the entropy concept of information theory, which measures the amount of information in an attribute.

In another embodiment of this aspect, the building of a decision tree includes the steps of: (i) obtaining a training set comprising a plurality of pro-haptens with a known rank-order; (ii) building a model based on the training set; (iii) testing the model on a second set comprising a plurality of pro-haptens; and (iv) comparing the testing results to results of other tests already deployed.

In another embodiment of this aspect, the multivariate analysis further includes selecting features using Support Vector Machine (SVM) and constructing a decision boundary that maximizes the marginal distance between the sensitizing and non-sensitizing classes, wherein a highly different profile in one or more of the metrics indicates the compound tested is a potential sensitizer.

In another embodiment of this aspect, the number of pro-haptens with a known rank-order as training set is at least about 5 or about 10, and the number of pro-haptens for testing the model is at least about 10 or about 20.

In another embodiment of this aspect, the creating of a predictive sensitization metric set includes the steps of (i) determining dominant parameters from a large data set and (ii) generating a metric using non-linear regression and the dominant parameter data in conjunction with an output classifier from a training set.

In another embodiment of this aspect, the comparing step further includes identifying distinguishing attribute(s) or feature(s) of the sample tested, and the classifying steps includes determining the classification of the tested sample based on its distinguishing attribute(s) or feature(s).

In another embodiment of this aspect, the method further includes exposing skin tissues to known sensitizing chemicals and measuring secretion of cytokines at different chemical concentrations; and visualizing the differences of the skin tissues to the chemicals.

In another aspect the present invention provides an immune modeling system for predicting potency of skin sensitizers, the system containing: a) a viable epidermis to provide barrier function and skin metabolism; and b) a dendritic cell compartment, wherein dendritic cells are activated.

In one embodiment of this aspect, the immune modeling system further contains c) a T cell compartment that allows for T cell activation by migrating, activated dendritic cells.

In another embodiment of this aspect, the dendritic cells (DCs) are Langerhans cells.

In another aspect the present invention provides a method of assessing in vivo skin sensitizing activity of a compound, including the steps of: (a) culturing cells of an in vitro cell model in a medium; (b) adding a test compound at a concentration to the culture medium comprising the cells; (c) measuring secretion level of one or more cytokine markers of the cells; (d) analyzing correlation of the concentration applied in step (b) with the secretion level measured in step (c); and (e) determining in vivo sensitization value based on the analysis of step (d).

In one embodiment of this aspect, the culturing step further includes (i) inducing differentiation of the cells with one or more differentiation cytokines, and (ii) inducing maturation with one or more maturation cytokines.

In another embodiment of this aspect, the culturing step is conducted in multi-well transwell chambers having an upper chamber and a lower chamber, the upper and lower chambers separated by a filter through which the cells can migrate from one chamber to another, wherein one or more chemokines are present or absent in the lower chamber.

In another embodiment of this aspect, the method further includes the steps of (i) measuring the number of cells migrated into the lower chamber, and (ii) calculating fold increase in migration.

In another embodiment of this aspect, the method further includes a Fluorescence Activated Cell Sorting (FACS) analysis of the cells, the analysis including the steps of (i) staining undifferentiated cells with mouse IgG1 anti-human fluorescein antibody, (ii) incubating for a period of time (for example, for about 30 minutes), and (iii) analyzing expression of a marker selected from CD80, CD83, CD86, CD54, CCR7, CD207, CD14, and CD11c using flow cytometry.

In another embodiment of this aspect, the culturing step further includes a functional analysis using a multiplex cytokine analyzer, the analysis including: (i) collecting supernatants of undifferentiated, differentiated, and mature cells; (ii) testing the supernatants for cytokines with respective standards to detect cytokine secretion levels; and (iii) optionally converting the cytokine secretion levels from [pg/mL] to [pg/million cells/day] by normalizing with the cell number for each cell stage of differentiation, wherein media samples are used as controls and supplementation with growth factors and cytokines are taken into account in the calculations.

In another embodiment of this aspect, the in vitro cell model is a Mutz3 phenotype or Episkin™.

In another embodiment of this aspect, the test compound is applied to the cells in varying dosage amounts or concentrations.

In another embodiment of this aspect, the cytokine markers are selected from the group consisting of: IL-1ra, IL-2, IL-4, IL-5, IL-6, IL-7, IL-8, IL-9, IL-10, IL-12, IL-13, IL-15, IL-17, eotaxin, bfGF, G-CSF, GM-CSF, IFN-γ, IP-10, MCP-1(MCAF), CCL3, CCL4, PDGF-bb, TNF-a, VEGF, and IL8.

In another embodiment of this aspect, the method further includes: (i) incubating the test compound with cultured cells in a trans-well chamber to allow for binding; and (ii) measuring the amount of secreted cytokines.

In another aspect the present invention provides a method for determining the sensitizing activity of a compound, comprising: (a) incubating a test compound with cells to allow for binding; (b) measuring the amount of secreted cytokines; and (c) comparing the secretion profile of the cytokines with a training set of known sensitizers.

In one embodiment of this aspect, the cytokine marker is selected from IL-1ra, IL-2, IL-4, IL-5, IL-6, IL-7, IL-8, IL-9, IL-10, IL-12, IL-13, IL-15, IL-17, eotaxin, bfGF, G-CSF, GM-CSF, IFN-γ, IP-10, MCP-1(MCAF), CCL3, CCL4, PDGF-bb, TNF-a, VEGF, and IL8.

In another embodiment of this aspect, the test compound is applied in varying dosage amounts or concentrations.

The advantages of the present invention are illustrated below in conjunction with the corresponding Figures.

### Local Lymph Node Assay (LLNA)

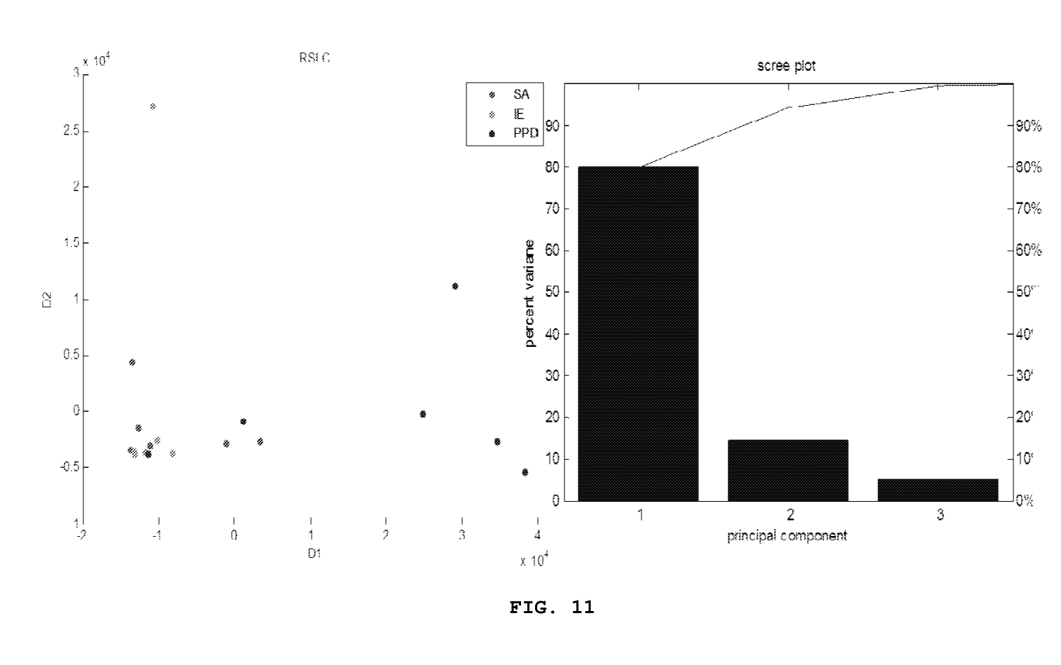

As illustrated in FIG. 10, a decision tree was constructed according to the traditional method utilized by the field and in literature in order to classify 211 chemicals into the following categories: weak, moderate, strong, extreme, and non-sensitizers. The metrics that were used to construct this classifier are molecular weight (MW), skin penetration coefficient (log Kp), and octanol-water partition coefficient (log Ko/w), all of which were calculated from the DEREK expert system.

The decision tree demonstrates that the aforementioned metrics do not provide a reliable means to classify the chemicals, as indicated by the numerous branches that identify the same class. This classifier is over fitting the data and will not generalize well to unknown chemicals. Hence, an alternative approach is needed, which requires first to identify metrics that have better predicative power.

### Principal Component Analysis (PCA)

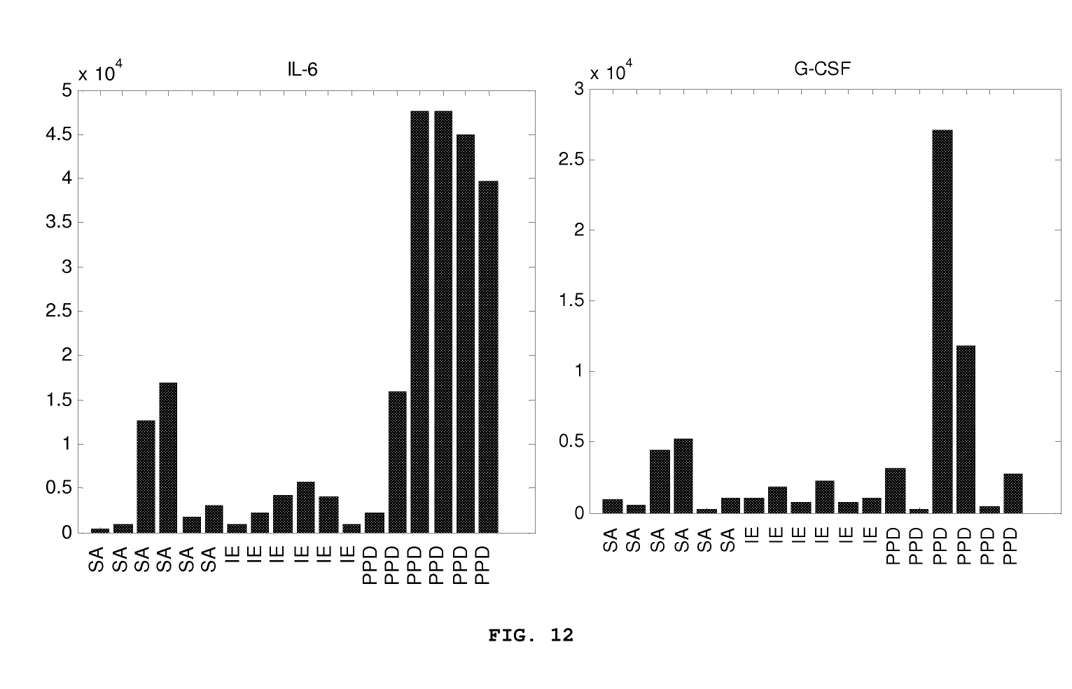

We have taken cytokine measurements of skin tissues that were exposed to known sensitizing chemicals. This data consists of 27 cytokines (27 dimensions) from several chemical concentrations and multiple replicates. Principal component analysis (PCA) was used to reduce the high dimensional data into a lower dimension so that the differences in response from each chemical could be visualized. The PCA result (also a limited, traditional approach) in FIG. 11 shows that the chemicals do not induce distinct cytokine profiles, especially between isoeugenol (IE, a strong sensitizer) and sialic acid (SA, a non-sensitizing irritant).

PCA reduces the dimension by projecting the data unto orthogonal axes that have the highest variance. This projection, however, does not take into account the difference between intra- and inter-class variance. FIG. 12 selectively shows two of the 27 dimensions (IL6 and G-CSF) that contribute to the most variance. Yet the inter-class difference between SA and IE is unclear. This led us to investigating other methods that would select features that have good inter-class variance.

### Quadratic Discriminant Analysis (QDA)

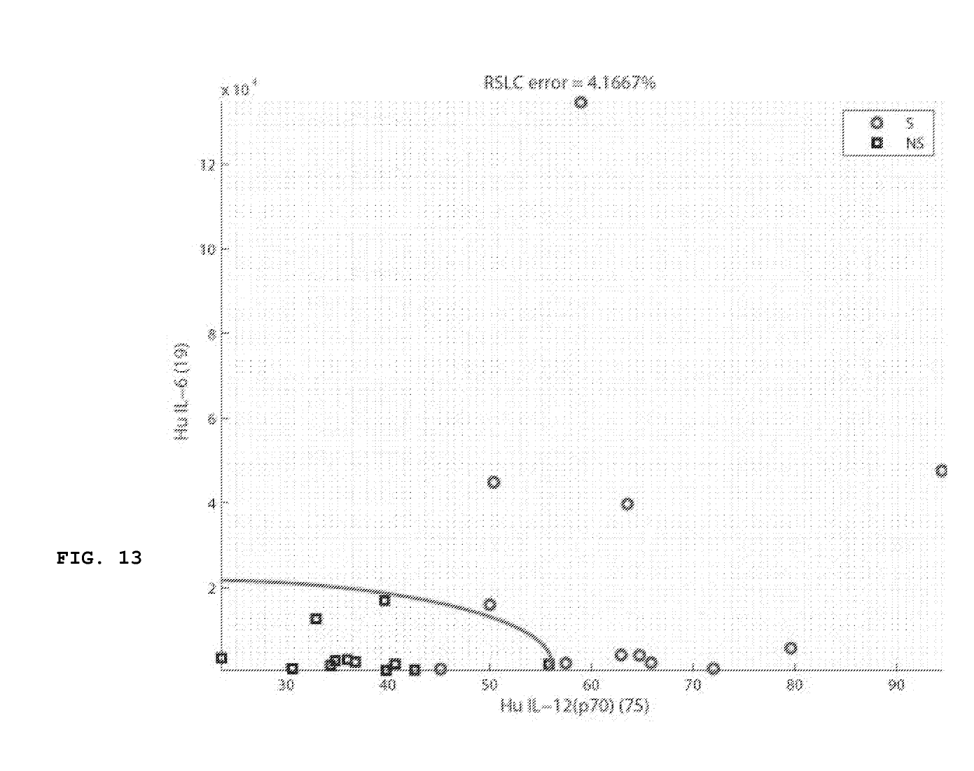

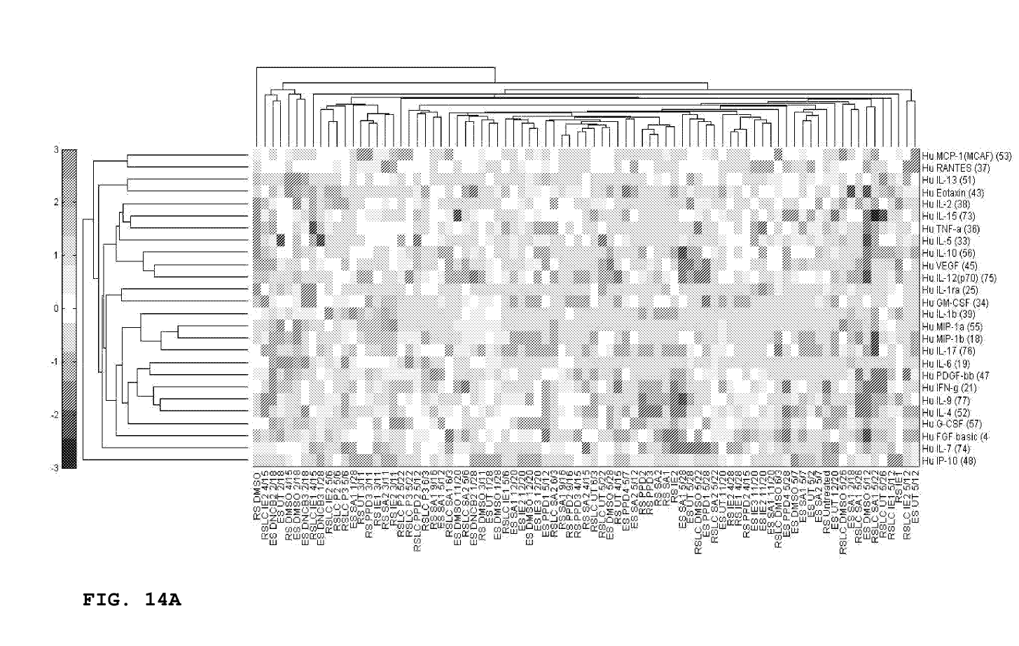

Quadratic discriminant analysis (QDA) uses a likelihood ratio test to calculate a boundary that separates the classes. It assumes that both classes are normally distributed. We tested all the combinations of two features and calculated the resubstitution error for each. The combination that had the lowest error was IL6 and IL12 as shown in FIG. 13.

The limitations of using this method for feature selection include 1) the assumption of normal distributions, and 2) only comparison of two features at a time.

### Hierarchy Clustering

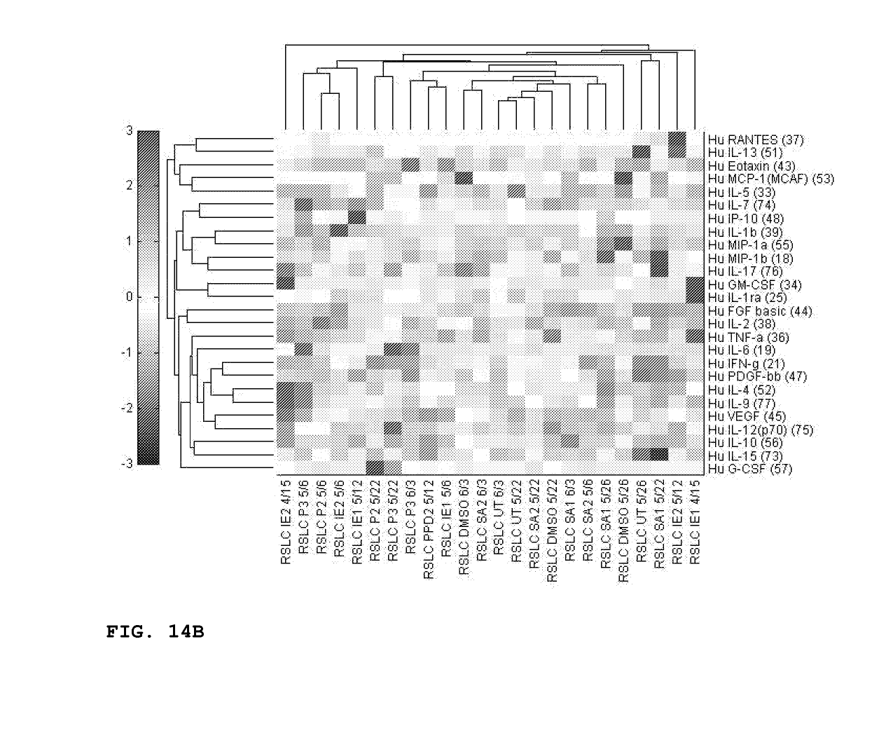

As shown in FIG. 14, which contains the hierarchical clustering of cytokine profiles from the full skin set and the reduced set of RSLC only, hierarchical clustering groups features together based on how close they are from each other, using Eucledian distance. The clusters are ranked, with the closest features clustered in the lowest brackets, and merge as the hierarchy goes up. This is simply another way of showing the overlap that exists in most of the cytokine profiles, and the need to extract discriminant features.

### Support Vector Machine (SVM)

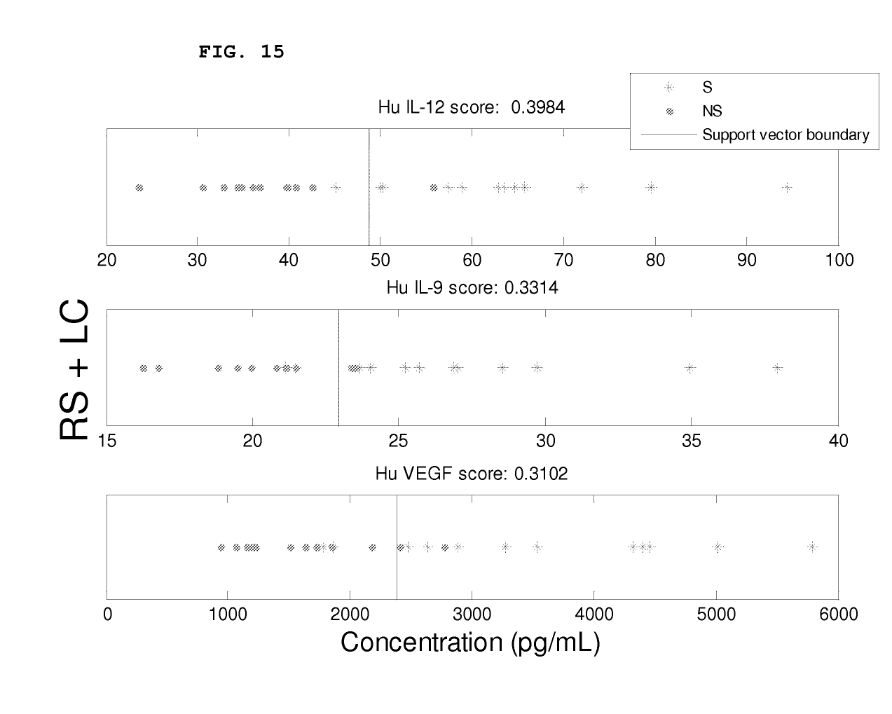

Support vector machine (SVM) is an optimization program that computes the hyperplane that maximizes the distance of points on either side of the plane. In one embodiment of the present invention, use of SVM for feature selection is illustrated in FIG. 15. This approach gives us the following benefits: 1) rank features using the separation distance, which is optimal because the further apart the chemicals are represented by a biological marker, the less likely this marker will give false positive result; 2) the boundaries can be used as a classifier to classify unknown chemicals; and 3) no assumption about the underlying distributions of the data.

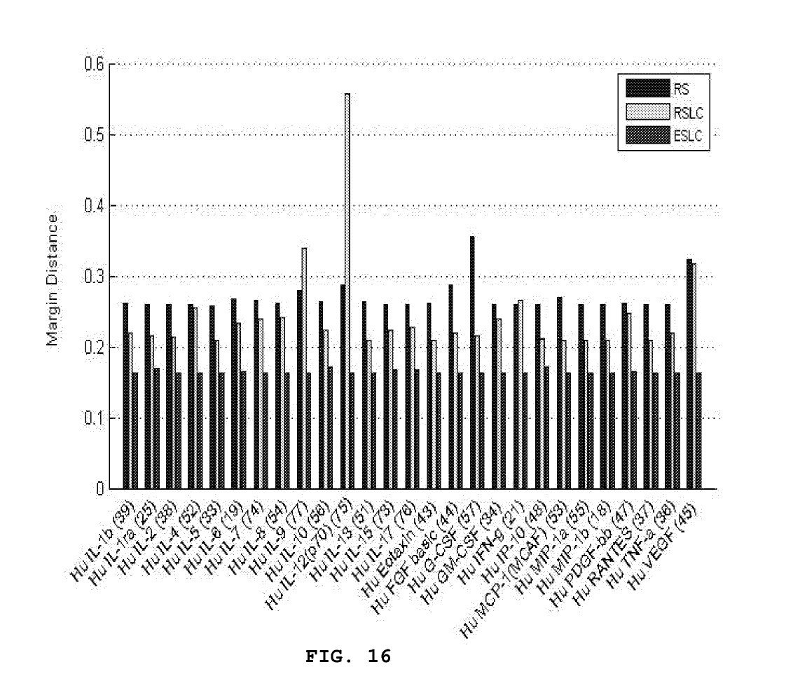

In another embodiment of the present invention, as illustrated in FIG. 16, the SVM feature selection is used for the full ranking of all cytokines in different skin types. A higher margin distance implies better predicative power.

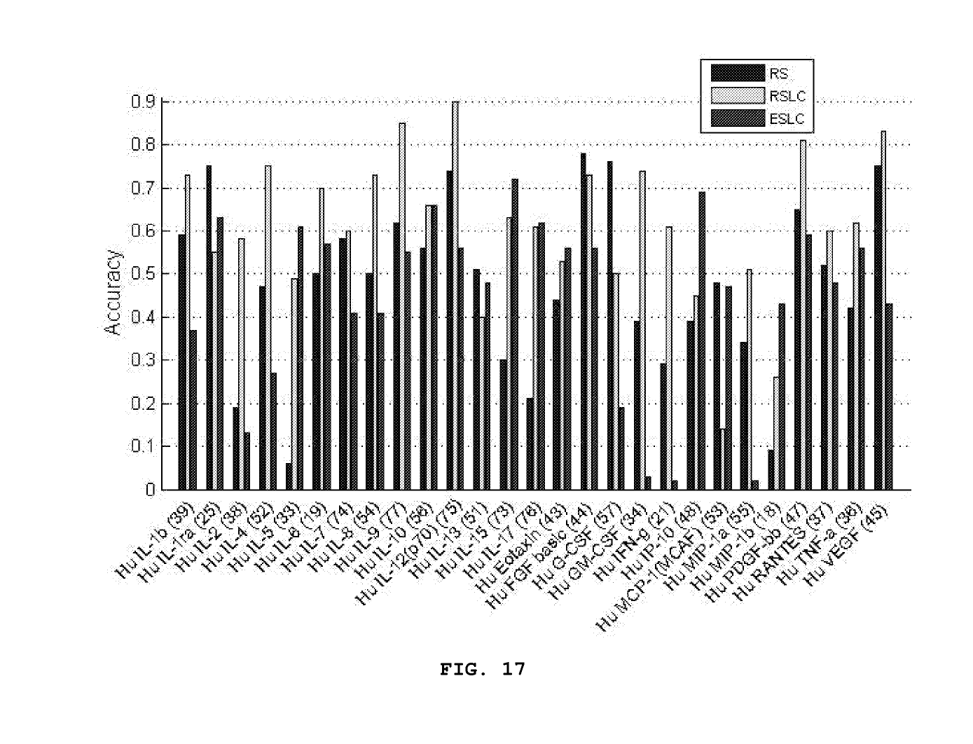

In another embodiment of the present invention, as illustrated in FIG. 17, the “leave-one-out” cross validation is used to test the accuracy of the boundaries generated in SVM. The accuracy score can then be used to combine the cytokine scores to generate a weighted classifier that takes both the margin distance and accuracy into account.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, biochemistry, or analytical chemistry described herein are those well known and commonly used in the art. The techniques and procedures are generally performed according to conventional methods well known in the art and as described in various general and more specific references that are cited and discussed throughout the present specification or available to a person skilled in the art. See, e.g., Sambrook et al. Molecular Cloning: A Laboratory Manual (2nd ed., Cold Spring Harbor Laboratory Press, N.Y. (1989) and Coligan et al., Current Protocols in Immunology (Wiley Interscience (1994)), which are incorporated herein by reference.

The present invention provides methods for predicting the in vivo skin sensitizing activity of chemical compounds using a combination of in vitro cell models with a multivariate analysis. In particular, the present invention provides in vitro screening methods by detecting the secretion of cytokine markers associated with skin sensitization. In preferred embodiments, these methods involve assays to measure the secretion of key cytokine(s) associated with skin sensitization in the in vitro cell models, coupled with multivariate analysis, and correlation with concentration or dosage. These methods can provide a means of high-throughput assessment of the potential of chemical compounds that can potentially act as skin sensitizers. The cytokines associated with skin sensitization used in the present invention include, but are not limited to, IL-1ra, IL-2, IL-4, IL-5, IL-6, IL-7, IL-8, IL-9, IL-10, IL-12, IL-13, IL-15, IL-17, eotaxin, bfGF, G-CSF, GM-CSF, IFN-γ, IP-10, MCP-1(MCAF), CCL3, CCL4, PDGF-bb, TNF-a, VEGF, and IL8.

In one aspect the present invention provides use of cytokines as markers for skin sensitization in a high-throughput manner. The use of these markers to assess skin sensitization in the human cell line model of Mutz3 phenotypes, combined with computational analysis to predict an in vivo sensitizing activity, has not been reported. The accuracy of the in vitro prediction of in vivo sensitizing activity is improved by using training set compounds of known potencies. Furthermore, a database of known skin sensitizers can be used to compare the effects of unknown chemicals and to provide important perspective with regard to predicting in vivo sensitizing activity.

### Prediction of In Vivo Skin Sensitization

In particular aspects, these assays will involve culturing in vitro cell models, including but not limited to Mutz3 phenotypes, in a culture medium containing various a chemical compound with varying concentrations; measuring the secretion level of one or more cytokine markers associated with skin sensitization in response to culturing in various (at least two, preferably at least three) concentrations of the chemical compound at the undifferentiated, differentiated and mature cell stages and predicting sensitizing activity of these chemical compounds from such measurements. The various embodiments involved in conducting such assays are described in further detail below. This method should also be applicable to other cell models, including but not limited to human keratinocytes (HaCat cells), 3D human skin cells, normal human epithelial cells (NHEK cells), MCF7 cells, H4IIE cells, or combination cultures with keratinocyte and dendritic cells.

The foregoing method requires preparing cell cultures. Such a cell may be a primary cell in culture or it may be a cell line. The cells could also be obtained from any mammalian source that is amenable to primary culture and/or adaptation into cell lines. In lieu of generating cell lines from animals, such cell lines may be obtained from, for example, American Type Culture Collection, (ATCC, Rockville, Md.), or any other Budapest treaty or other biological depository.

The cells used in the assays are preferably derived from tissue obtained from humans. Techniques employed in mammalian primary cell culture and cell line cultures are well known to those of skill in that art. In the case of commercially available cell lines, such cell lines are generally sold accompanied by specific directions of growth, media and conditions that are preferred for that given cell line.

Various concentrations of the chemical compound being tested are added to each cell media and the cells are allowed to grow exposed to the various concentrations of a test chemical compound. Furthermore, the cells may be exposed to the test chemical compound at any given phase in the growth cycle. For example, in some embodiments, it may be desirable to contact the Mutz3 cells or Episkin™ with the compound at the same time as a new cell culture is initiated.

### Predicting In Vivo Skin Sensitizing Activity of a Compound From In Vitro Analyses

Once all data for secretion of one or more cytokine markers in cultured cells in culture media containing various concentrations of the chemical compound are received, the data are analyzed to obtain a detailed profile of the compounds toxicity. Higher concentrations or longer exposure times may decrease cell viability to a level where accurate assessment and evaluation of the marker genes cannot be determined

In certain embodiments, the determination of a predicted in vivo sensitization value comprises performing concentration response analyses of measurements from at least three separate assays for each cytokine marker in each of the cell line.

Distinguishing Sensitization from Irritation

Of particular value to the present invention is IL-8, a stress-induced cytokine that provides an indication of cellular stress that may not be linked to sensitization. The inclusion of IL-8 provides a means of identifying potential irritants. Inclusion of IL-8, as well as other genetic markers such as IL-1, IL-6, and TNF alpha, makes it possible to differentiate chemicals that cause irritation but not sensitization (see US 2009/0305276).

### EXAMPLES

The present invention is further illustrated by the following examples, which should not be construed as limiting in any way. While some embodiments have been illustrated and described, it should be understood that changes and modifications can be made therein in accordance with ordinary skill in the art without departing from the invention in its broader aspects as defined in the following claims.

### Description of Process

Limitations inherent in traditional approaches have been first outlined using a dataset of 211 compounds, as shown in FIGS. 1-5. Our approach utilizes support vector machines (SVM). In brief, for each marker (cytokines, cell surface markers, chemical metrics, migration rate), the training data is given by {xᵢ,yᵢ}, i=1 . . . l, yᵢ∈{−1,1}, where −1 denotes non-sensitizers and 1 denotes sensitizers. Using a linear kernel, the points that lie on the hyperplane that separates the positives and negatives is w·x+b=0, which leads to the following constraints to the training data:

xᵢ·w+b≧+1 for yᵢ=+1 (1)

xᵢ·w+b≦−1 for yᵢ=−1 (2)

where w is the norm to the hyperplane and

$$\frac{\vert b \vert}{‖ w ‖}$$

is the perpendicular distance from the hyperplane to the origin, and ∥w∥ is the Eucledian norm of w.

Suppose that the points in equality (1) holds, the perpendicular distance of the hyperplane to the origin is

$$\frac{\vert 1 - b \vert}{‖ w ‖} .$$

Similarly, the distance of the hyperplane in (2) to the origin is

$$\frac{\vert - 1 - b \vert}{‖ w ‖} .$$

Therefore the margin distance between these two hyperplanes is

$$\frac{\vert b \vert}{‖ w ‖} .$$

Thus we can find a pair of hyperplanes that best separates the two classes by minimizing ∥w∥ subject to (1) and (2).

The Lagrangian can be expressed as:

$${min}_{w , b} {max}_{α} \{ \frac{1}{2} {‖ w ‖}^{2} - {\sum }_{i = 1}^{n} {α}_{i} [ {y}_{i} ( w \cdot {x}_{i} - b ) - 1 ] \}$$

which is then solved using quadratic programming provided by the Bioinformatics Toolbox, Matlab.

By using this method we computed w for each cytokine, and then ranked them by their margin distance 2/∥w∥.

### Reagents and Supplies

Culture media, glutamine, penicillin/streptomycin, 2-mercaptoethanol and fetal bovine serum were purchased from Invitrogen technologies (Carlsbad, Calif.). rhGMCSF, hTNF-alpha, hTGFbeta1, hIL-6 and hIL-1beta were purchased from R&D systems. PGE₂ was purchased from Sigma. Mutz3 cells were a gift from L'Oreal (Paris, France). 5637 urinary bladder carcinoma cell line was purchased from ATCC (VA). Episkin™, a skin composite consisting of human keratinocytes on bovine collagen I with a thin layer of human collagen IV was purchased from L'Oreal (Paris, France). Salicylic acid and isoeugenol were purchased from Sigma. Nylon discs were purchased from Small Parts Inc.

### Mutz3 Cell Culture

Mutz3 cells were cultured in routine format in T-75 flasks at an initial density of 1.5 million cells per flask. Culture media is composed of alpha-MEM with Glutamax, ribonucleosides [Invitrogen] and deoxyribonucleosides supplemented with 20% FBS, 1% Penicillin/Streptomycin and 10% conditioned medium. 50 uM of freshly prepared 2-Mercaptoethanol was added to each flask during media changes and cell splitting. Cultures were passaged every 5 days with media changes every two days. Passages 37-48 were used for the culture, differentiation and maturation experiments.

Conditioned medium was prepared from human urinary bladder carcinoma 5637 cell line. To establish cultures, cells were plated at a density of 5×10⁵ cells/ml in a T-75 flask in Advanced RPMI medium supplemented with 10% FBS, 4 mM glutamine and 1% Penicillin/Streptomycin. Forty eight (48) hours after plating, cell media was changed and approximately 42-43 hours after the media change, conditioned medium was collected. The media was filtered and stored at −80° C. before utilization for preparation of Mutz3 proliferation medium.

### Mutz3 Cell Differentiation and Maturation

To induce differentiation, Mutz3 cells were cultured for 7 days in T-75 flasks (10⁵ cells/mL, 20 ml medium per flask) in complete α-MEM medium with the following cytokines: 100 ng/ml GM-CSF, 2.5 ng/ml TNF-α and 10 ng/mL TGF-β1. At D2 and D5 fresh cytokines equivalent to 10 ml of medium were added in each flask. On day 7, MUTZ3-LCs were harvested, spun down and re-seeded in 12 well plates (2×10⁵ cells/mL, 5 ml medium per T-25 flask) in complete α-MEM medium (without 5637) with the following maturation cytokines mix 100 ng/mL IL6, 50 ng/mL TNFα, 25 ng/mL IL1β and 1 μg/mL PGE₂.

### Migration of Mutz3 Cell Lineages in Transwell Chambers

mMutz-LCs were plated at a density of 5×10⁴ cells/well in an 8 um pore 24-well transwell insert in the presence and absence of chemokine CCL19, CCL21 or CXCL12 in the lower chamber. Four (4) hours after exposure, the migrated Mutz3 cells were counted in the lower chamber. For cell number measurements, cells post-migration were centrifuged and resuspended in 100 ul of fresh media to increase cell density. Also, the number of migrated cells is determined as follows:

{Net # of cells migrated}={# of cells migrated in presence of chemokine}−{# of cells migrated in absence of chemokine}

$$Fold Increase in Migration = \frac{[ Net Migration for Sensitizer Treated Cells ]}{[ Average Net Migration for Irritant Treated Cells ]}$$

Fluorescence Activated Cell Sorting (FACS) analysis of Mutz3 cells

Approximately 1-5 million undifferentiated Mutz3 cells, Mutz3-LCs and mature Mutz-LCs were stained with mouse IgG1 anti-human fluorescein antibody and incubated for 30 mins followed by expression analysis using flow cytometry for the following markers: CD80, CD83, CD86, CD54, CCR7, CD207, CD14 and CD11c. All fluorescein conjugated antibodies were purchased from R&D systems.

### Functional Analysis Using Multiplex Cytokine Analyzer

Multiple cytokines and growth factors were assessed using the Biorad Bioplex Analyzer. 1 ml supernatants were collected for undifferentiated Mutz3 cells, Day 7 differentiated cells [Mutz3-LC] and Day 9 mature cells [mMutz3-LC]. The supernatants were tested for 27 cytokines with respective standards to detect cytokine secretion levels. Basal media samples were utilized as controls and supplementation with growth factors and cytokines viz. GMCSF, TGF-beta1, TNF-alpha, IL-6, IL1β were taken into account in the calculations. Cytokine secretion levels [pg/ml] were converted to [pg/million cells/day] by normalizing with the cell number for each cell stage of differentiation.

### Mutz3-LC Sensitization

Different doses of SA (Salicylic Acid), IE (Isoeugenol), PPD (Paraphenylenediamine) and DNCB (DiNitroChlorobenzene) were topically applied onto a 1 cm² nylon disc to Episkin™ in 12 well inserts. The lower chamber consisted of Mutz3-LCs at a density of 250,000 cells/ml. Forty eight (48) hrs post sensitization, cells were resuspended in 1 ml fresh maturation media, supernatants were collected for subsequent analysis and cell count was performed. A portion of the cells were tested using FACS for CD54 and CD86 and comparison between different conditions was performed using a stimulation index as below.

% Stimulation Index is calculated as

$$\% Simulation Index = \frac{[ MFI * Percent Positive Cells for Treated Condition ]}{[ MFI * Percent Positive Cells for Untreated Condition ]} * 100$$

where MFI corresponds to Mean Fluorescence Intensity of cell condition for a particular phenotypical marker.

$$Fold Increase in Surface Marker Expression = \frac{[ \% Simulation Index for Sensitizer Treated Condition ]}{[ \% Simulation Index for Irritant Treated Condition ]}$$

The remaining cells were tested for migration in response to chemokine CCL19.

The foregoing detailed description and drawings describe and illustrate various exemplary embodiments. The description and drawings serve to enable one skilled in the art to make and use the invention, and are not intended to limit the scope of the invention in any manner.

1. A method of assessing in vivo skin sensitizing activity of a compound, comprising: (a) culturing cells of an in vitro cell model in a medium; (b) adding a test compound at a concentration to the culture medium comprising the cells; (c) measuring secretion level of one or more cytokine markers of the cells; (d) analyzing correlation of the concentration applied in step (b) with the secretion level measured in step (c); and (e) determining in vivo sensitization value based on the analysis of step (d).

2. The method of claim 1, wherein said culturing further comprises (i) inducing differentiation of the cells with one or more differentiation cytokines, and (ii) inducing maturation with one or more maturation cytokines

3. The method of claim 1, wherein said culturing is conducted in multi-well transwell chambers comprising an upper chamber and a lower chamber, the upper and lower chambers separated by a filter through which the cells can migrate from one chamber to another, wherein one or more chemokines are present or absent in the lower chamber.

4. The method of claim 3, further comprising (f) measuring the number of cells migrated into the lower chamber; and (g) calculating fold increase in migration.

5. The method of claim 1, further comprising Fluorescence Activated Cell Sorting (FACS) analysis of the cells, the analysis comprising: staining undifferentiated cells with mouse IgG1 anti-human fluorescein antibody, incubating for a period of time (about 30 minutes), and analyzing expression of a marker selected from CD80, CD83, CD86, CD54, CCR7, CD207, CD14, and CD11c using flow cytometry.

6. The method of claim 1, wherein said culturing further comprises a functional analysis using a multiplex cytokine analyzer, the analysis comprising: (i) collecting supernatants of undifferentiated, differentiated, and mature cells; (ii) testing the supernatants for cytokines with respective standards to detect cytokine secretion levels; and (iii) optionally converting the cytokine secretion levels from [pg/mL] to [pg/million cells/day] by normalizing with the cell number for each cell stage of differentiation, wherein the culture medium samples are used as controls and supplementation with growth factors and cytokines are taken into account in the calculations.

7. The method of claim 1, wherein the in vitro cell model is a Mutz3 phenotype or Episkin™.

8. The method of claim 1, wherein the cells are Mutz3, Mutz3-LC, or mMutz3-LC cells.

9. The method of claim 1, wherein the test compound is applied to the cells in varying dosage amounts or concentrations.

10. The method of claim 1, wherein the cytokine markers are selected from the group consisting of: IL-1ra, IL-2, IL-4, IL-5, IL-6, IL-7, IL-8, IL-9, IL-10, IL-12, IL-13, IL-15, IL-17, eotaxin, bfGF, G-CSF, GM-CSF, IFN-γ, IP-10, MCP-1(MCAF), CCL3, CCL4, PDGF-bb, TNF-a, VEGF, and IL8.

11. A method for assessing potency of skin sensitizers, comprising: (a) measuring one or more co-culture metrics of post-sensitization skin or artificial skin/dentric cells; (b) analyzing the co-culture metrics using a computational algorithm; and (c) comparing the analysis results with those of a set of known sensitizers.

12. The method of claim 11, wherein the co-culture metrics are selected from cytokine profiles, chemotaxis, cell migration, cell surface expression, and intracellular protein expression.

13. The method of claim 11, wherein said measuring comprises determining the changes of the metrics in temporal response to different chemical stimuli.

14. The method of claim 11, wherein said measuring comprises a high-throughput data acquisition through testing a plurality of stimuli compounds in a plurality of wells simultaneously.

15. The method of claim 11, wherein the plurality is 12, 24, 48, or 94.

16. The method of claim 11, wherein the measuring comprises detecting cytokine or growth factor secretion profile of a Mutz3 phenotype selected from Mutz3, Mutz3-LC, and mMutz3-LC, wherein the cytokine or growth factor is involved in contact hypersensitivity.

17. The method of claim 16, wherein the cytokine or growth factor is selected from the group consisting of IL-1ra, IL-2, IL-4, IL-5, IL-6, IL-7, IL-8, IL-9, IL-10, IL-12, IL-13, IL-15, IL-17, eotaxin, bfGF, G-CSF, GM-CSF, IFN-γ, IP-10, MCP-1(MCAF), CCL3, CCL4, PDGF-bb, TNF-a, VEGF, and IL8.

18. The method of claim 16, wherein the cytokine or growth factor is selected from the group consisting of IL-1ra, IFN-γ, CCL3, CCL4, IL-2, IL-4, IL-17, Eotaxin, bFGF, PDGF-bb, and IL-8.

19. The method of claim 11, wherein the analyzing comprises determining a ranking system for chemical stimuli tested using a multivariate analysis.

20. The method of claim 19, wherein the multivariate analysis comprises:
(a) creating a predictive sensitization metric set;
(b) building a decision tree from a training set of examples;
(c) comparing attribute(s) of a test sample with the predictive metric set; and
(d) classifying the tested sample based on comparison results and the decision tree.

21. The method of claim 20, wherein the decision tree comprises a plurality of leaf nodes and a plurality of non-leaf nodes; and the leaf nodes comprise class names, and a non-leaf node is a decision node, the decision node being an attribute test and comprising a plurality of branches, with each branch being a possible value of the attribute and connected to another decision tree.

22. The method of claim 20, wherein the predictive sensitization metric set is created using an ID3 program comprising: (i) using a statistical property known as information gain to help decide which attribute goes into a decision node, and (ii) defining information gain using the entropy concept of information theory, which measures the amount of information in an attribute.

23. The method of claim 20, wherein said building a decision tree comprises (i) obtaining a training set comprising a plurality of pro-haptens with a known rank-order; (ii) building a model based on the training set; (iii) testing the model on a second set comprising a plurality of pro-haptens; and (iv) comparing the testing results to results of other tests already deployed.

24. The method of claim 20, wherein the multivariate analysis further comprises selecting features using support vector machine (SVM) and constructing a decision boundary that maximizes the marginal distance between the sensitizing and non-sensitizing classes, wherein a substantially different profile in one or more of the metrics indicates the compound tested is a potential sensitizer.

25. The method of claim 23, wherein said plurality of pro-haptens with a known rank-order is at least about 5 or about 10, and said plurality of pro-haptens in step (ii) is at least about 10 or about 20.

26. The method of claim 20, wherein said creating a predictive sensitization metric set comprises (i) determining dominant parameters from a data set; and (ii) generating a metric using non-linear regression and the dominant parameter data in conjunction with an output classifier from a training set.

27. The method of claim 20, wherein said comparing further comprises identifying distinguishing attribute(s) or feature(s) of the sample tested, and said classifying comprises determining the classification of the tested sample based on its distinguishing attribute(s) or feature(s).

28. The method of claim 20, further comprising exposing skin tissues to known sensitizing chemicals and measuring secretion of cytokines at different chemical concentrations; and visualizing the differences of the skin tissues to the chemicals.
